(19) United States
(12) Patent Application Publication
(10) Pub. No.: US 2024/0008839 A1
(43) Pub. Date: Jan. 11, 2024

(54) ARTIFICIAL INTELLIGENCE-TYPE THERMAL IMAGING ULTRASOUND SCANNER APPARATUS FOR BREAST CANCER DIAGNOSIS USING SMART MIRROR, AND BREAST CANCER SELF-DIAGNOSIS METHOD USING SAME

(71) Applicant: RESEARCH & BUSINESS FOUNDATION SUNGKYUNKWAN UNIVERSITY, Suwon-si (KR)

(72) Inventor: Jae-Chern YOO, Suwon-si (KR)

(73) Assignee: RESEARCH & BUSINESS FOUNDATION SUNGKYUNKWAN UNIVERSITY, Suwon-si (KR)

(21) Appl. No.: 18/253,666

(22) PCT Filed: Mar. 2, 2022
(86) PCT No.: PCT/KR2022/002933

§ 371 Date: May 19, 2023

(30) Foreign Application Priority Data
Mar. 22, 2021 (KR) 10-2021-0036638

(51) Int. Cl. A61B 8/08; A61B 5/01; A61B 8/00; A61B 5/1171; G16H 50/20; G16H 30/40

(52) U.S. Cl. CPC A61B 8/0825; A61B 5/015; A61B 8/4263; A61B 8/54; A61B 8/4472; A61B 8/463; A61B 8/40; A61B 5/1176; G16H 50/20; G16H 30/40

(57) ABSTRACT

Disclosed herein is a device of self-diagnosing breast cancer with an artificial intelligence and with no assistance from a physician, the present disclosure provides a thermographic ultrasound scanner device that guides a proper posture correction of an ultrasound scanner with an augmented reality that delivers feedback control commands to a patient who self-diagnoses through a smart mirror and enables a patient to self-diagnose breast cancer by an artificial intelligence neural network that has been learned by thermographic and ultrasound images, and a method of self-diagnosing using the same.

### TECHNICAL FIELD

The present disclosure relates to a device for self-diagnosing breast cancer with an artificial intelligence and with no assistance from a physician, more particularly to a thermographic ultrasound scanner device having a pressure sensor to measure contact pressure between an ultrasound probe and the affected area, and configured to guide a correct posture correction of an ultrasound scanner by an augmented reality that delivers feedback control commands to a patient who self-diagnoses using a smart mirror with an object image, and enables a patient to self-diagnose breast cancer using an artificial intelligence neural network that has been learned by a thermographic image and an ultrasound image, and a method of self-diagnosing using the same.

### BACKGROUND ART

Recently, digital image processing technology has been applied to the field of clinical diagnosis along with medical equipment manufacturing technology, which has led to many advances in radiology.

In particular, an ultrasound diagnosis avoids harmful radiation exposure compared to CT or X-ray medical equipment, and has the characteristics of obtaining cross-sectional images of the human body in a non-invasive method, as well as being portable and low cost. In particular, the ultrasound diagnosis has the advantage of obtaining images in real time, thus observing a state of organ movement in real time.

These ultrasound diagnostic technology is widely used for breast cancer screening along with mammography using X-rays.

However, an improper positioning of the ultrasound scanner (tilt, angle of incidence, contact pressure, position, etc.) results in low resolution and noise in the ultrasound image, which reduces the quality of the ultrasound image for breast ultrasound diagnosis. In addition, since the ultrasound scanner requires different contact pressure and tilt for different locations, it is practically impossible to handle unless an experienced professional. In this reason, it is difficult to self-test at home.

Furthermore, breast ultrasound images alone are inaccurate in detecting breast cancer, so mammography is often used in conjunction with ultrasound to diagnose breast cancer. However, mammography is a radiation-exposing procedure that uses X-rays. Therefore, it is impossible for patients to use mammography for self-diagnosis.

Meanwhile, scientists have found that breast cancer is associated with an increase in skin temperature in the affected area. However, the screening for breast cancer using thermography based on skin temperature measurements may lead to many misdiagnoses and not be applicable as a standard screening method. However, thermography is still of interest to scientists, the medical device industry, and patients because thermography is a non-contact method of measurement.

Breast cancer is a highly curable disease when detected at an early stage, and as the importance of self-diagnosis for breast cancer has increased worldwide, many self-diagnosis methods have been widely known. However, since self-diagnosis is not made using specialized equipment, there have been many problems with measurement accuracy and systematic data management.

The background art of the present application is disclosed in Korean Patent Application Laid-Open No. 10-2020-0144821.

### DISCLOSURE

### Technical Problem

According to the technical solutions of the present application, an object of the present disclosure is to provide a thermographic ultrasound scanner device that uses thermographic and ultrasound images together for breast cancer screening to improve diagnostic accuracy and enable self-diagnosis.

For example, a patient determines signs of breast cancer using a thermographic camera and a thermographic artificial intelligence neural network installed on a smart mirror while the patient normally looks at the smart mirror. When breast cancer is suspected, the smart mirror may request the patient to re-diagnose the breast cancer using an ultrasound scanner for the suspected breast cancer area (breast cancer hot spot).

In this case, the present disclosure has an effort to provide a thermographic ultrasound scanner device and a method of self-diagnosing breast cancer which is capable of self-diagnosing breast cancer by an ultrasound artificial intelligence neural network that has been learned with ultrasound images, while guiding a patient to correct posture of the ultrasound scanner by feedback control command means using augmented reality in order to provide the patient with optimal posture information (incident angle, pressure, and position) of the ultrasound scanner.

However, technical problems to be solved by the exemplary embodiment of the present application are not limited to the aforementioned technical problem, and other technical problems may be present.

### Technical Solution

As a technical solution for achieving the above-mentioned technical objects, an aspect of the present disclosure provides an artificial intelligence thermographic ultrasound scanner device that includes a smart mirror and a ultrasound scanner.

According to an embodiment of the present disclosure, in a mode in which the ultrasound scanner is operating, diagnosing a disease may be performed using the ultrasound scanner while observing an image of a patient reflected in a mirror of the smart mirror, but which is not limited thereto.

According to an embodiment of the present disclosure, the smart mirror may include: a thermographic imaging camera configured to detect thermal radiation emitted from a patient's body to obtain a two-dimensional thermographic image; a plurality of image sensors configured to obtain a body image of the patient; a wireless receiver configured to receive ultrasound image information collected from the ultrasound scanner; a speaker configured to identify posture information of the ultrasound scanner, and deliver feedback control commands to the patient to guide an optimized posture of the ultrasound scanner for each examination site, or provide the patient with guidance and instructions necessary for the ultrasound diagnosis; a display panel configured to identify posture information on the ultrasound scanner, and to deliver the feedback control commands through a virtual object image to guide a optimal posture of the ultrasound scanner to the patient by an inspection area, or to inform the guidance and instructions required for the ultrasound diagnosis through the virtual object image; a virtual object imaging unit configured to generate the virtual object image on the display panel; a thermographic imaging breast cancer detector configured to detect a breast cancer hot spot, which is an area suspected of having breast cancer, from the thermographic image; and an ultrasound artificial intelligence neural network that has been pre-learned by breast cancer ultrasound images for training, labeled according to a rating of risk level of breast cancer and configured to determine signs of breast cancer from the ultrasound image information, in which the ultrasound scanner may include: an ultrasound probe configured to obtain the ultrasound image information from the affected area in contact with a patient's breast; and a wireless transmitter configured to transmit the ultrasound image information to a wireless receiver of the smart mirror, in which the smart mirror displays the virtual object image on the display panel with the virtual object image overlapping on an image of the patient reflected in the mirror, and analyzes the ultrasound image information obtained from the patient by the ultrasound artificial intelligence neural network to automatically determine the risk of breast cancer of the patient, but which is not limited thereto.

According to an embodiment of the present disclosure, the thermographic breast cancer detector comprising: a breast thermographic image mapper configured to generate a breast thermographic image consisting of an average value of a pixel-by-pixel cumulative sum of thermographic images of a breast area taken equal to or more than a predetermined number of times over a predetermined period of time; a cutoff value adjuster configured to generate a thermographic hot spot image comprising a breast cancer hot spot that represents a temperature value equal to or more than a predetermined value in the breast thermographic image; and a breast cancer hot spot memory configured to store the thermographic hot spot image or breast cancer hot spot information, but which is not limited thereto.

According to an embodiment of the present disclosure, the smart mirror may further include a hot spot guider configured to inform an area and position of the breast cancer hot spot obtained from a thermographic camera mode during an ultrasound scanner mode or a self-examination mode in augmented reality by overlapping the object image to the mirror on which the patient's image is projected, but which is not limited thereto.

According to an embodiment of the present disclosure, the smart mirror may further include a pressure sensing artificial intelligence neural network that has been pre-learned by breast ultrasound images labeled according to magnitudes of various pressure levels and determine a magnitude of a pressure level of the ultrasound scanner from breast ultrasound images obtained during an ultrasound scanner mode, but which is not limited thereto.

According to an embodiment of the present disclosure, the smart mirror may further include a body posture interaction unit, in which the body posture interaction unit may include: an angle of incidence calculator 93 configured to calculate a position of the ultrasound scanner and a position and incident angle of the ultrasound scanner by the image sensor, and provide position and incident angle correction information of the ultrasound scanner; a body navigator configured to create a body map which comprises boundary lines to distinguish body parts and a body outline from the body image; a body posture correction requester configured to provide a body posture to be taken by the patient during a breast cancer diagnosis by a virtual object image configured to show a body outline image for posture fitting, a body fitting identifier configured to calculate the degree of fitting between the body outline image of the patient and the body outline image for posture fitting and provide feedback to the patient; and a patient verification unit configured to verify who the patient is, but which is not limited thereto.

According to an embodiment of the present disclosure, the patient verification unit may be a face recognition unit that is configured to recognize who a face is by comparing an image of a facial part on the body map with a face database of pre-registered patients, but which is not limited thereto.

According to an embodiment of the present disclosure, the body navigator may include an artificial intelligence neural network that has been learned by deep learning from body images labeled with semantic segmentation in different colors for body parts, and obtain the body map using the artificial intelligence neural network for a body image of a given patient from the image sensor, but which is not limited thereto.

According to an embodiment of the present disclosure, the body navigator may include an artificial intelligence neural network that has been learned by deep learning from body images labeled with a body outline image that comprises boundary lines separating body parts, and obtain the body map using the artificial intelligence neural network for a body image of a given patient from the image sensor, but which is not limited thereto.

According to an embodiment of the present disclosure, the smart mirror may include a standing position information providing means configured to calculate optimal position information favorable (corresponding) to a breast cancer diagnosis during a thermographic camera mode and provide the patient with the position information, but which is not limited thereto.

According to an embodiment of the present disclosure, the position information providing means may display a body outline image for fitting or a footprint outline for fitting which indicates a standing place favorable for diagnosing breast cancer by measuring a height of the patient by the image sensor through the virtual object image on the display panel, or provide a standing place by a laser beam scanning means, but which is not limited thereto.

According to an embodiment of the present disclosure, the virtual object image may be any one or more object images selected from a virtual cursor, pressure correction information, incident angle correction information, a breast outline, a body outline, a footprint outline for fitting, boundary lines distinguishing body parts, and a breast cancer hot spot, but which is not limited thereto.

According to an embodiment of the present disclosure, the hot spot guider, during an ultrasound scanner mode or a self-examination mode, may display the breast cancer hot spot on the display panel by overlapping the breast cancer hot spot on the patient's breast image reflected in the mirror in augmented reality to guide the patient to re-examine the breast cancer hot spot area, but which is not limited thereto.

According to an embodiment of the present disclosure, the smart mirror may further include a breast cancer tracking and management unit configured to perform periodic ultrasound follow-up examinations to observe changes in size of a tumor, lump, or calcification cluster over time to inform the patient of a risk level, or to additionally register a tumor, lump, or calcification cluster detected during an ultrasound scanner mode in the breast cancer hot spot memory, or to inform the patient of a schedule for the next breast cancer ultrasound examination, but which is not limited thereto.

According to an embodiment of the present disclosure, the smart mirror may further include a breast cancer tracking and management unit configured to observe changes in the size and number of breast cancer hot spots based on periodic follow-up examinations by the thermographic camera to inform the patient of a risk level or to inform the patient of a schedule for the next breast cancer thermographic camera examination, but which is not limited thereto.

A method of self-diagnosing breast cancer according to another aspect of the present disclosure, which is performed by the artificial intelligence thermographic ultrasound scanner device, the method including: providing a patient with optimal position information favorable (corresponding) to diagnosing breast cancer by a standing position information providing means; finding a breast cancer hot spot by a thermographic camera; requesting an ultrasound examination or self-examination to the patient when the breast cancer hot spot is detected by the thermographic camera; providing posture correction information on the ultrasound scanner with a virtual object image on the smart mirror during an ultrasound scanner mode; displaying the breast cancer hot spot found during self-diagnosis on the smart mirror by overlapping the breast cancer hot spot on an image of a patient reflected in the mirror in augmented reality; performing periodic ultrasound follow-up examinations by a breast cancer tracking and management unit to observe changes in size of a tumor, lump, or calcification cluster over time to inform the patient of a risk level of breast cancer or a schedule for the next breast cancer examination; and observing changes in the size and number of breast cancer hot spots based on the periodic follow-up examinations with the thermographic camera by the breast cancer tracking and management unit to inform the patient of a risk of breast cancer or a schedule for the next breast cancer examination.

The technical solution is just illustrative but should not be interpreted as being intended to limit the present application. In addition to the above-mentioned exemplary embodiment, additional exemplary embodiments may be present in the drawings and the detailed description of the invention.

### Advantageous Effects

According to the technical solution of the present application that has been described above, the present disclosure provides a thermographic ultrasound scanner device that guides a proper posture correction of an ultrasound scanner with an augmented reality that delivers feedback control commands to a patient who self-diagnoses through a smart mirror, and enables a patient to self-diagnose breast cancer by an artificial intelligence neural network that has been learned by thermographic and ultrasound images, and a method of self-diagnosing using the same.

According to the technical solution of the present application that has been described above, the thermographic ultrasound scanner according to the present disclosure not only enables a patient to perform frequent breast cancer examinations in the comfort of the patient's own home without any exposure to radiation, but also significantly improves reliability of breast cancer examinations by securing big data on the patient as the number of self-examinations increases.

However, the effects, which can be obtained by the present application, are not limited to the above-mentioned effects, and other effects may be present.

### DESCRIPTION OF DRAWINGS

FIG. 1 is a view illustrating an implemented example of a thermographic ultrasound scanner device according to an embodiment of the present disclosure.

FIG. 2 is a view illustrating an operating principle of the thermographic ultrasound scanner device according to an embodiment of the present disclosure.

FIG. 3 is a view illustrating embodiments of a body navigator for obtaining a body map from a body image in the thermographic ultrasound scanner device according to an embodiment of the present disclosure.

FIG. 4A is a view illustrating embodiments of a position information providing means for providing a patient with position information on an optimal standing position favorable for breast cancer diagnosis when the patient stands in front of a smart mirror, using the thermographic ultrasound scanner device according to an embodiment of the present disclosure.

FIG. 4B is a view illustrating an embodiment of the position information providing means having patient footprint position identification means of a thermographic ultrasound scanner device, according to an embodiment of the present disclosure.

FIGS. 5A to 5C are views illustrating virtual object images of the thermographic ultrasound scanner device, according to an embodiment of the present disclosure, showing virtual cursors, pressure correction information, and incident angle correction information during an ultrasound scanner mode.

FIGS. 6A and 6B are embodiments of performing ultrasound self-diagnosis using the smart mirror of the thermographic ultrasound scanner device according to an embodiment of the present disclosure, by overlaying a virtual object image necessary for diagnosing breast cancer on a display panel over the patient's own image reflected in the mirror.

### BEST MODE

Hereinafter, exemplary embodiments of the present disclosure will be described in detail with reference to the accompanying drawings so that those with ordinary skill in the art to which the present application pertains may easily carry out the exemplary embodiments. However, the present disclosure may be implemented in various different ways, and is not limited to the exemplary embodiments described herein. Apart irrelevant to the description will be omitted in the drawings in order to clearly describe the present application, and similar constituent elements will be designated by similar reference numerals throughout the specification.

Throughout the specification of the present application, when one constituent element is referred to as being “connected to” another constituent element, one constituent element can be “directly connected to” the other constituent element, and one constituent element can also be “electrically connected to” or “indirectly connected to” the other element with other elements therebetween.

Throughout the specification, when one member is disposed “on”, “at an upper side of”, “at an upper end of”, “below”, “at a lower side of”, or “at a lower end of” another member in the present specification of the present application, this includes not only a case where one member is brought into contact with another member, but also a case where still another member is present between the two members.

Throughout the specification of the present application, unless explicitly described to the contrary, the word “comprise” or “include” and variations, such as “comprises”, “comprising”, “includes” or “including”, will be understood to imply the inclusion of stated constituent elements, not the exclusion of any other constituent elements.

Throughout the specification of the present application, the tilt of an ultrasound probe is used interchangeably with the tilt of an ultrasound scanner.

Throughout, the tilt of the ultrasound scanner is used interchangeably with the incident angle, which is an angle at which the ultrasound probe faces the surface of the patient.

Throughout the specification of the present application, the patient is used interchangeably with self-diagnosing party.

Throughout the specification of the present application, an ultrasound scanner mode refers to a period of time when an ultrasound scanner is used to obtain a breast ultrasound image from a patient.

Throughout the specification of the present application, a thermographic camera mode refers to a period of time during which the thermographic camera is operating to obtain a corresponding thermographic image from a patient's breast.

Throughout the specification of the present application, self-examination mode refers to a period of time during which a patient examines her own breasts by touching her own breasts using the breast self-examination method while looking at the smart mirror and with the help of virtual object images and audio information provided by the smart mirror.

Throughout the specification of the present application, the breast self-examination method includes a process of examining a list of self-examination items in detail by the patient, which includes bruising and pain, the presence of lumps, nipple discharge, nipple depression, breast wrinkles, nipple eczema, changes in breast skin, changes in breast size, and changes in nipple position.

The breast self-examination method may include a process of answering questions by a digital ARS provided by the smart mirror.

For example, the digital ARS may ask the patient whether the nipple discharge is present, and the patient may answer “yes” or “no”.

The question and answer of the digital ARS according to the present disclosure may be performed by a question and answer sentence provided as an object image on a display panel and a click window for selecting an answer by touching in addition to a voice message.

Throughout the specification of the present application, the self-diagnosis includes a thermographic camera mode, an ultrasound scanner mode, and a self-examination mode.

Throughout the specification of the present application, posture information of the ultrasound scanner according to the present disclosure refers to information indicating an incident angle, contact pressure, and position of the ultrasound scanner with respect to the affected area, and posture correction information of the ultrasound scanner refers to an incident angle correction, a pressure correction, and position information of the ultrasound scanner that is required to maintain an optimized ultrasound scanner posture according to a diagnostic area based on the posture information of the ultrasound scanner.

The smart mirror according to the present disclosure is a display device that combines a mirror and a touch display panel, and is manufactured by attaching a mirror film to the touch display panel. The smart mirror has the form of a mirror, but the smart mirror is a mirror to pursue the convenience of self-diagnosis by displaying object images that provide various functions such as time, weather, date, and medical guide service for self-diagnosis on the display panel, such that a reflection of a user in the mirror is overlapped with the object image.

In addition to the above-describe functions, the smart mirror may provide an Augmented Reality function for applications in the beauty industry, such that, when a user selects a desired color, the smart mirror shows the user that hair and lips are dyed in the desired color by overlaying the desired color on the user's own image, or before buying clothes, shows a desired fashion by overlaying the desired fashion on the user's own image.

The present disclosure is provided to solve the problems of the related prior art described above. In a thermographic ultrasound scanner device according to the present disclosure, a patient uses an ultrasound scanner while looking at the patient's own reflection in a smart mirror during an ultrasound scanner mode. The smart mirror may include: a thermographic camera configured to detect thermal radiation emitted from a patient's body to obtain a two-dimensional thermographic image; a plurality of image sensors configured to obtain a body image of the patient; a wireless receiver configured to receive ultrasound image information collected from the ultrasound scanner; a speaker and a display panel configured to identify posture information of the ultrasound scanner, and deliver feedback control commands to the patient to guide an optimized posture of the ultrasound scanner for each examination site, or provide the patient with guidance and instructions necessary for the ultrasound diagnosis; a breast thermographic image mapper configured to obtain a breast thermographic image showing a temperature distribution of the breast from the thermographic image; and an ultrasound artificial intelligence neural network previously deep-learning learned by breast cancer ultrasound images for training.

The ultrasound scanner may include: an ultrasound probe configured to obtain the ultrasound image information from the affected area in contact with a patient's breast; and a wireless transmitter configured to transmit the ultrasound image information to a wireless receiver of the smart mirror.

The smart mirror takes the ultrasound image information obtained from a patient and analyzes the ultrasound image information using the ultrasound artificial intelligence neural network to automatically determine a risk level of breast cancer of the patient.

The wireless transmitting and receiving connections according to the present disclosure are preferably performed by Wi-Fi, Bluetooth, or Internet of Things connections.

The display panel of the smart mirror of the present disclosure overlaps a virtual object image required to diagnose breast cancer on a patient's own reflection in the mirror to show a progression for the patient performing the self-diagnosis.

It is preferred that the breast thermographic image mapper generates a breast thermographic image consisting of an average value of a pixel-by-pixel cumulative sum of thermographic images of a breast area taken equal to or more than a predetermined number of times over a predetermined period of time.

By using an average value of the thermographic images of a breast area obtained equal to or more than a predetermined number of times, reliability of the breast cancer test is much better than a result of a thermographic image taken only once a year.

Hereinafter, an image consisting of image pixels (breast cancer hot spot) indicating a temperature value equal to or more than a predetermined value in the breast thermographic image is referred to as a thermographic hot spot image, and the area of the corresponding pixels is referred to as a breast cancer hot spot area.

The hot spot area of breast cancer obtained from the thermographic camera mode refers to an area with a higher temperature compared to other areas, and this area corresponds to a suspected area in which breast cancer occurs.

Accordingly, the thermographic ultrasound scanner device of the present disclosure provides a means capable of detecting breast cancer at an early stage by reexamining the breast cancer hot spot area obtained in the thermographic camera mode in the ultrasound scanner mode or in the self-examination mode by the patients themselves, thereby increasing reliability of the breast cancer examination.

In addition, the smart mirror includes a hot spot guider that informs the breast cancer hot spot area and position obtained from the thermographic camera mode during the ultrasound scanner mode or self-examination mode by overlapping the object image to the mirror on which the patient's own image is projected, thereby allowing interactive diagnosis with the patient.

For example, during the self-examination mode, patients may look in the mirror and touch an area on their breast that corresponds to the breast cancer hot spot area indicated by the hot spot guider to identify whether there are any lumps or bumps.

In another aspect, the thermographic hot spot image may be obtained from the breast thermographic image via an artificial intelligence neural network that has been pre-learned using the breast thermographic image semantically segmented and labeled by the breast cancer hot spots.

In the present disclosure, the breast cancer ultrasound images for training preferably consist of ultrasound images labeled by the grades of breast cancer. For example, the breast cancer ultrasound images for training may be categorized into normal, mild, moderate, and severe grades and labeled by the grades of breast cancer.

In addition, the ultrasound scanner may further include a pressure sensor mechanically coupled to the ultrasound probe that measures how strongly the ultrasound probe is squeezed to scan the patient's affected area to generate pressure information.

In this case, the ultrasound image information preferably includes the measured pressure information along with the ultrasound image and is transmitted to the wireless receiver of the smart mirror.

The pressure sensor of the present disclosure refers to a sensor that measures a pressure level that indicates how much pressure the ultrasound probe is pressing against the affected area when the ultrasound probe is in contact with the affected area. Maintaining a contact pressure determined by clinical experience (e.g., a standardized pressure) depending on a diagnostic position is required to obtain a good ultrasound image. It is preferred to use any one of the pressure sensors selected from resistance film pressure sensors, piezoelectric pressure sensors, and resistance strain gauge type pressure sensors.

In another aspect of the pressure sensor according to the present disclosure, the pressure sensor includes a pressure sensing artificial intelligence neural network that has been learned by breast ultrasound images labeled according to various pressure levels. Thereafter, it is preferred that the pressure sensor uses the learned pressure sensing artificial intelligence neural network to determine a pressure level of the ultrasound scanner from the breast ultrasound images obtained during the ultrasound scanner mode to provide pressure correction information.

Additionally, the smart mirror may further include a body navigator that generates a body map including a body outline and a boundary line distinguishing the patient's face, breast, arm, stomach, leg, foot, and other body parts on the patient's body image. Therefore, on the body map obtained by the body navigator, a breast outline may be obtained that distinguishes the breast from other body parts.

The body map may be obtained by the artificial intelligence neural network that has been learned by deep learning from body images labeled with Semantic Segmentation in different colors for face, breast, arm, stomach, leg, foot, and the rest of the body parts.

From the semantically segmented body image, the boundary lines that distinguish the body parts and the body outline may be obtained. For example, a boundary between a semantically segmented belly and breast provides a breast boundary line (breast outline).

The semantic segmentation is an artificial intelligence neural network that categorizes pixel-by-pixel where a specific object is positioned in a given image, and separates the specific object from other objects.

In another aspect of the body navigator according to the present disclosure, the body navigator may include an artificial intelligence neural network that has been learned by body images labeled with body outline images that include boundary lines separating body parts including face, breast, arm, stomach, leg, and foot, and the body navigator may obtain a body map using the artificial intelligence neural network for a given patient's body image from the image sensor.

In another aspect of the body navigator according to the present disclosure, the body navigator is implemented by the body map in which, on the body outline image, boundary lines separating major body parts are disposed (included) in consideration of medical and physical disposition correlations.

The body outline image may be obtained by acquiring a differential image between the image when the patient is not in front of the smart mirror and the image when the patient is in front of the smart mirror, and taking an edge component thereof.

In this case, the body map is obtained by placing boundary lines separating the body parts on the body outline image based on the medical and physical disposition of the body parts (e.g., face, arms, breast, stomach, legs, feet, etc.) and statistical body parts disposition method for the size ratio between the body parts.

Preferably, the statistical body part disposition method selects positions of body parts according to medical statistics based on the patient's physical information including sex, race, age, height, weight, and waist circumference entered upon patient registration.

It is preferred that the patient authentication is performed by any one of the methods selected from face recognition, fingerprint recognition, voice recognition, and ID authentication that are registered upon patient registration.

The smart mirror according to the present disclosure may further include a face recognition unit that compares an image of a facial part on the body map with a face database of a pre-registered patient to recognize who the corresponding face is.

The smart mirror is provided with a body posture correction requestor that provides the patient with a body posture that the patient needs to take using an object image to facilitate the diagnosis of the patient's breast cancer.

For example, in case that the body posture correction requester needs to examine the patient's armpit, it is preferred to guide the patient to raise both arms, display the raised arms as an object image using a body outline image for posture fitting, calculate a degree of fitting between the patient's body outline image and the body outline image for posture fitting, and provide feedback to the patient.

The body outline image for posture fitting of the body posture correction requestor is preferred to be a body outline image of a standing posture while the patient is looking straight ahead toward the smart mirror.

It is preferred that the degree of fitting between the body outline image of the patient and the body outline image for posture fitting is fed back to the patient using an object imaging means.

In addition, the smart mirror may further include an angle of incidence calculator configured to calculate a position of the ultrasound scanner and an incident angle of the ultrasound scanner by the image sensor during the ultrasound scanner mode.

In this case, it is preferred that the smart mirror uses two or more image sensors, and it is even more preferred that the image sensors are installed on the left and right sides of the smart mirror to calculate the incident angle and position of the ultrasound scanner. Therefore, the image sensors disposed on the left and right sides of the smart mirror provide a stereo vision to recognize the three-dimensional information of objects.

A self-diagnosing person may adjust the incident angle of the ultrasound scanner to improve quality of the ultrasound image acquired from the affected area.

The smart mirror further includes a standing position information providing means configured to calculate and provide optimal standing position information favorable for diagnosing breast cancer to the patient during the thermographic camera mode.

In order to detect breast cancer with the thermographic camera, the patient needs to stand in an optimal position in consideration of the field of view (FOV) and focal length of the thermographic camera.

The optimal standing position information refers to an optimal place where the patient needs to stand in front of the smart mirror for breast cancer screening by the thermographic camera, i.e., a standing place.

It is preferred that the standing position information providing means measures the height of the patient by the image sensor and displays a standing place favorable for breast cancer diagnosis or the body outline image for posture fitting through the virtual object image on the display panel.

In another aspect, the standing position information providing means may provide the standing place as footprint position information by a laser beam scanning means.

For example, the laser beam scanning means may form a footprint-like laser footprint pattern on the floor surface of the standing place to provide footprint position information.

It is preferred that the standing position information providing means according to the present disclosure provides the patient with a standing position during the ultrasound scanner mode or self-examination mode in the same position as used in the thermographic camera mode.

Further, the standing position information providing means further includes a patient footprint position identification means configured to identify whether a sole of the patient's foot is within the range of the laser footprint pattern by the image sensor.

For example, the patient footprint position identification means may detect a foot on the body map and identify whether there is a laser footprint pattern in the corresponding area or a degree of fitting with the foot to determine how well the patient fits and aligns with the standing place.

Furthermore, it is preferred that the speaker and display panel of the smart mirror provide an audio or visual guide to the patient to fit the patient's foot within the range of the laser footprint pattern.

In addition, it is preferred that the laser footprint pattern blinks when the patient's foot remains outside the range of the laser footprint pattern.

The display panel of the smart mirror according to the present invention shows a progress to the patient performing the self-diagnosis by an augmented reality that overlaps a virtual object image required to diagnose breast cancer on the patient's reflection in the mirror.

It is preferred that the display panel according to the present disclosure utilizes a transparent a thin film transistor liquid crystal display (TFT-LCD) or a transmissive organic light emitting diode (OLED).

The transparent display panel is transparent, making it easier for the image sensor and thermographic camera to measure the patient beyond the display panel.

In addition, the display panel and mirror film of the smart mirror may be made of a material selected from Germanium, Chalcogenide, and Zinc Selenide (ZnSe) to make the light in the infrared band pass through well at a lens opening position of the thermographic camera, and an opening may be installed in a part of the display panel that is optically aligned with the lens of the thermographic camera, if necessary.

It is preferred that the virtual object image includes virtual body parts during the thermographic camera mode.

It is preferred that the virtual object image according to an embodiment of the present disclosure is one or more object images selected from a virtual cursor, pressure correction information, incident angle correction information, virtual body part, and breast cancer hot spot.

The virtual body parts include the breast outline and the body outline of the patient.

The virtual cursor overlaid on the patient's own reflection in the mirror is a reference for the patient to calibrate the position of the ultrasound scanner with respect to the breast cancer hot spot, the pressure correction information displayed in the mirror is a reference for the patient to calibrate the pressure of the ultrasound scanner, and the “incident angle correction information” overlaid on the reflection of the ultrasound scanner in the mirror is a reference for the patient to calibrate the incident angle of the ultrasound scanner.

The breast cancer hot spot area and position obtained during the thermographic camera mode are calculated based on the breast outline. It is preferred to store the calculated breast cancer hot spot area and position in a breast cancer hot spot memory.

The hot spot guider may, during the ultrasound scanner mode or the self-examination mode, display the breast cancer hot spot on the display panel by overlapping the breast cancer hot spot on the patient's breast image reflected in the mirror in augmented reality, thereby guiding the patient to intensively re-examine the breast cancer hot spot area.

For example, during the ultrasound scanner mode, the hot spot guider may display the breast cancer hot spot obtained during the thermographic camera mode on the display panel in augmented reality by overlapping the breast cancer hot spot on the patient's breast image in the mirror to guide the patient to be re-examined by the ultrasound scanner for the breast cancer hot spot.

To this end, based on the breast outline during the ultrasound scanner mode, the breast cancer hot spot area and position obtained in the thermographic camera mode are aligned and overlapped on the patient's breast reflected in the mirror through the object image and displayed in augmented reality.

In addition, it is preferred that during the self-examination mode, the hot spot guider overlaps the breast cancer hot spot obtained during the thermographic camera mode with the patient's breast image reflected in the mirror and presents the breast cancer hot spot to the patient in augmented reality on the smart mirror to guide the patient to repeatedly examine the breast cancer.

To this end, during the self-examination mode, the hot spot guider aligns a position of the breast cancer hot spot area obtained from the thermographic camera mode, relative to the breast outline in the self-examination mode, and displays the breast cancer hot spot area overlapped on the patient's breast image reflected in the mirror through the object image.

The virtual cursor represents the current position of the ultrasound scanner projected over the patient's reflection in the mirror during ultrasound scanner mode.

Further, it is preferred that the virtual cursor blinks when the virtual cursor is outside the breast cancer hot spot area, and that the virtual cursor stops blinking when a position of the virtual cursor matches within a predetermined range from a position coordinate of the breast cancer hot spot.

In this case, a self-diagnosing person may easily recognize that he/she has intuitively found the breast cancer hot spot area, which is advantageous to correct a position of the ultrasound scanner.

The self-diagnosis person may easily identify the current position of the ultrasound scanner by the virtual cursor, and understand which direction to move the ultrasound scanner in order to get to the breast cancer hot spot area by the breast cancer hot spot represented by the object image.

In this case, the self-diagnosing person may intuitively know how much the coordinates between the current virtual cursor position and the breast cancer hot spot match or mismatch, which is advantageous for correcting the position of the ultrasound scanner.

Further, it is preferred that the hot spot guide displays the breast cancer hot spot area which has been re-examined by the ultrasound scanner in blue color and the breast cancer hot spot area which has not been re-examined by the ultrasound scanner in red color.

It is preferred that the incident angle correction information of the present disclosure utilizes an angle of incidence correction arrow that includes upward, downward, and left-right directions. The incident angle correction information is a virtual object image that instructs a person performing self-diagnosis, using arrow directional instructions, how to reach a pre-empirically known ideal incident angle of an ultrasound scanner according to the examination position.

In the present disclosure, when the incident angle of the current ultrasound scanner is inconsistent with the required incident angle, it is preferred that an angle of incidence correction arrow in a direction that requires correction is displayed by blinking on the smart mirror to guide the patient to correct the incident angle.

In this case, it is easy for a self-diagnosing person to intuitively know whether the current incident angle of the ultrasound scanner matches or mismatches the required incident angle, which is advantageous for correcting the incident angle of the ultrasound scanner.

Pressure correction information of the present disclosure includes contact pressure information required by the ultrasound scanner and contact pressure information of the current ultrasound probe.

It is preferred that the required contact pressure uses an ideal contact pressure value of the ultrasound probe, which has been clinically determined in advance, depending on the diagnostic site.

In the present disclosure, it is preferred that the required contact pressure information is displayed on the smart mirror by a bar graph, a pie graph, or a numerical value, and the current contact pressure information of the ultrasound scanner is also displayed.

An ultrasound artificial intelligence neural network according to an embodiment of the present disclosure has been supervised learning by breast ultrasound images for training that are semantic segmentation labeled with different colors for tumors, masses, and micro-calcification clusters. Thereafter, a semantic segmentation is performed on the breast ultrasound image of the patient obtained from the ultrasound scanner to obtain a semantically segmented ultrasound image for tumors, masses, and micro-calcification clusters; and based on the extent of the tumors, masses, or calcification clusters found in the semantically segmented ultrasound image, a risk level is provided to the patient.

In another aspect, the ultrasound artificial intelligence neural network uses a convolutional neural network (CNN) that has been supervised learning by the breast ultrasound images for training labeled for tumors, masses, and micro-calcification clusters. Thereafter, an ultrasound image of the patient's breast obtained from the ultrasound scanner is applied as input to the CNN, which informs the patient of a risk level based on the extent of the tumor, mass, or micro-calcification cluster found.

In addition, it is preferred that the smart mirror is further provided with a breast cancer tracking and management unit that performs periodic ultrasound follow-up examinations to observe changes in the size of tumors, masses, or calcification clusters over time to inform the patient of a risk level or to inform the patient of the next breast cancer ultrasound examination.

In addition, the breast cancer tracking and management unit may further increase the breast cancer hot spot area based on results of tumor, mass or calcification clusters found during the ultrasound scanner mode by the ultrasound artificial intelligence neural network.

That is, it is preferred that the breast cancer tracking and management unit additionally registers the tumors, masses, or calcification clusters detected during the ultrasound scanner mode as breast cancer hot spots in the breast cancer hot spot memory, and include the added breast cancer hot spots in the periodic follow-up examination.

Further, it is preferred that the breast cancer tracking and management unit observes a trend of changes in the size and number of breast cancer hot spots according to the periodic follow-up examination by the thermographic camera to inform the patient of a risk level or to inform the patient of a schedule for the next breast cancer thermographic camera examination.

It is preferred that the artificial intelligence neural network of the present disclosure utilizes semantic segmentation, a convolutional neural network (CNN), or a recurrent neural network (RNN).

In the present disclosure, the artificial neural network is a neural network that allows deep learning training and includes a combination of any one or more layers or elements selected from convolution layer, pooling layer, ReLu layer, transpose convolution layer, unpooling layer, 1×1 convolution layer, skip connection, global average pooling (GAP) layer, fully connected layer, support vector machine (SVM), long short term memory (LSTM), atrous convolution, atrous spatial pyramid pooling, separable convolution, and bilinear upsampling. It is preferred that the artificial intelligence neural network is further provided with an operation unit for batch normalization operation at the front of the ReLu layer.

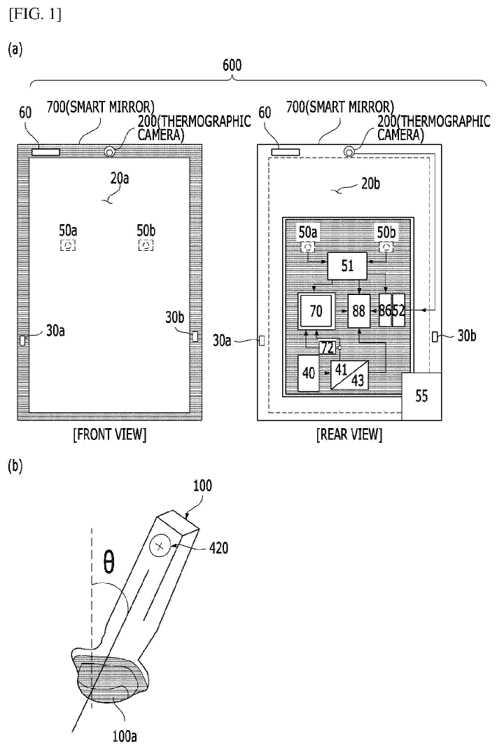

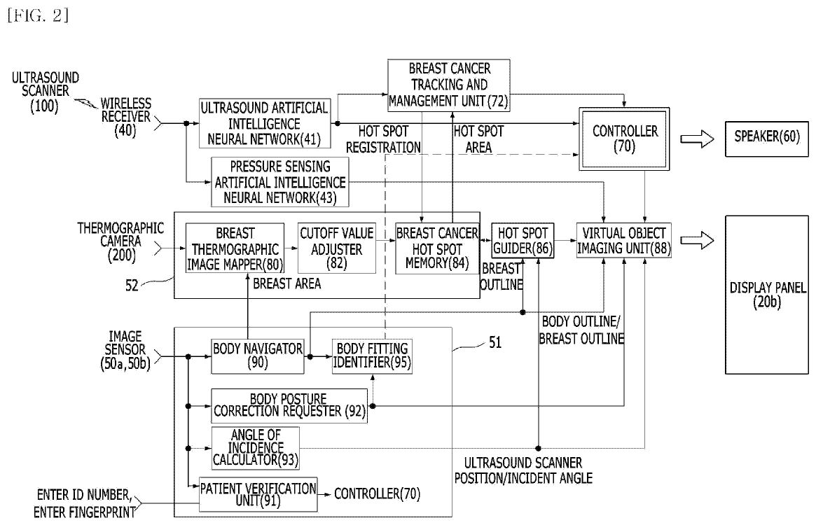

FIGS. 1 and 2 illustrate an embodiment of a thermographic ultrasound scanner device 600 using a smart mirror 700, which allows a patient to use an ultrasound scanner 100 while looking at the patient's own reflection in the smart mirror 700 during the ultrasound scanner mode.

The smart mirror 700 may include a thermographic camera 200 configured to obtain a thermographic image, which is a two-dimensional image of changes in infrared radiated according to a temperature distribution on a surface of a patient's affected area, and a plurality of image sensors 50a and 50b configured to obtain a body image of the patient.

In addition, the smart mirror 700 may include a wireless receiver 40 configured to receive ultrasound image information collected from the ultrasound scanner 100, a speaker 60 configured to identify posture information of the ultrasound scanner 100 and deliver feedback control commands to a patient to guide a standard posture of the ultrasound scanner optimized for each examination site, or to provide guidance and instructions to the patient for a breast cancer examination, and a display panel 20b.

In addition, the smart mirror 700 may include a thermographic breast cancer detector 52 that identifies a breast cancer hot spot, which is a suspected area of breast cancer, from the breast thermographic image, and a body posture interaction unit 51 that identifies a position and incident angle of the ultrasound scanner 100 by the image sensors 50a and 50b to provide position and incident angle correction information, or to create a body map with virtual body parts disposed on the body image, or to guide and feedback to the patient through an object image a body posture to be taken by the patient during a breast cancer diagnosis.

Further, the smart mirror 700 may include a virtual object imaging unit 88 configured to generate a virtual object image on the display panel 20b and an ultrasound artificial intelligence neural network 41 that has been pre-learned by breast cancer ultrasound images for training labeled with a rating of risk level of breast cancer based on a size and shape pattern of a tumor, lump, or calcification cluster that has developed in the breast to determine signs of breast cancer by the ultrasound images.

The ultrasound scanner 100 includes an ultrasound probe 100a configured to obtain ultrasound image information from the affected area by contact with a patient's breast, and a wireless transmitter 420 configured to transmit the ultrasound image information to the wireless receiver 40 of the smart mirror 700, in which the smart mirror 700 takes the ultrasound image information obtained from the patient and uses the learned ultrasound artificial intelligence neural network 41 to automatically determine a disease rating that indicates a risk level of breast cancer for the patient.

The smart mirror 700 according to the present disclosure is a display that combines a mirror 20a and the display panel 20b, and may show a patient performing a self-diagnosis a virtual object image which is overlapped on the patient's own reflection in the mirror 20a and necessary for a breast cancer examination on the display panel 20b in augmented reality.

The thermographic breast cancer detector 52 includes a breast thermographic image mapper 80 configured to obtain a breast thermographic image from the thermographic image, and a breast cancer hot spot memory 84 configured to store breast cancer hot spots that consist of pixels having a temperature value that is greater than a cutoff value determined by a cutoff level adjuster 82 on the breast thermographic image.

It is preferred that the breast cancer hot spot memory 84 stores breast cancer hot spot information or the thermographic hot spot image by itself.

The breast cancer hot spot information may include a center coordinate (a position) of the breast cancer hot spot, the breast cancer hot spot area, and their image pixel values.

It is preferred that the breast cancer hot spot area and center coordinate (position) obtained during the thermographic camera mode are calculated relative to the breast outline.

The breast thermographic image may be generated by the breast thermographic image mapper 80 as an average value of a pixel-by-pixel cumulative sum of thermographic images of the breast area taken at least a predetermined number of times over a predetermined period of time. To this end, it is preferred that the breast thermographic image mapper 80 aligns the thermographic images of the breast area in a two-dimensional space based on the breast outline, and then performs a pixel-by-pixel addition between the thermographic images and obtains the breast thermographic image as the average value of the addition.

The breast cancer hot spot area obtained in the thermographic camera mode refers to an area having a higher temperature compared to other areas, which may be selectively adjusted by the cut-off value adjuster 82.

When the patient performs a re-examination on the breast cancer hot spot area obtained in the thermographic camera mode by the ultrasound scanner mode or self-examination mode, the breast cancer examination may be more precise, thereby increasing accuracy of the examination and detecting the breast cancer at an earlier stage.

The hot spot guider 86 reads out the breast cancer hot spot obtained in the thermographic camera mode during the ultrasound scanner mode or the self-examination mode from the breast cancer hot spot memory 84, and displays the breast cancer hot spot on the display panel 20b in augmented reality overlapped by the virtual object imaging unit 88 on the mirror 20a in which the patient's own image is reflected.

For example, during the self-examination mode, a patient may perform a precise self-diagnosis of the presence of a lump by touching the breast cancer hot spot area while viewing an overlapping image between the breast cancer hot spot area indicated by the hot spot guide 86 and the breast area of the patient's own breast reflected in a mirror.

In this case, it is preferred that the hot spot guider 86 displays the breast hot spot area that has been examined by the self-diagnosis in a blue color, and the breast hot spot area that has not been re-examined by the self-diagnosis in a red color.

Therefore, the patient may perform an interactive breast cancer self-diagnosis based on augmented reality while being assisted by the smart mirror 700.

A reference numeral 43 is a pressure sensing artificial intelligence neural network configured to measure a pressure level that indicates how strongly the ultrasound scanner 100 is being pressed against the skin of a patient, which has been pre-learned by the breast ultrasound images for training labeled according to a magnitude of the pressure level. Thereafter, the pre-learned pressure sensing artificial intelligence neural network 43 may determine a magnitude of the current contact pressure level of the ultrasound scanner from the breast ultrasound images of the patient during the ultrasound scanner mode and provide the magnitude of the current contact pressure level of the ultrasound scanner to the virtual object imaging unit 88.

According to another embodiment for measuring the pressure level of the ultrasound scanner 100, a pressure sensor (not illustrated) may be provided within the ultrasound probe 100a to transmit measured pressure information to the wireless receiver 40 of the smart mirror, which may be used to determine the pressure level of the ultrasound scanner 100.

A body posture interaction unit 51 may include an angle of incidence calculator 93 configured to calculate a position of the ultrasound scanner 100 and an incident angle of the ultrasound scanner 100 by the image sensors 50a and 50b during the ultrasound scanner mode.

In addition, the body posture interaction unit 51 may include a body navigator 90 configured to create a body map by finding a body outline from a body image of a patient and generating boundary lines to distinguish body parts such as a face, breast, arm, stomach, leg, foot, etc. of the patient.

Further, the body posture interaction unit 51 includes a body posture correction requester 92 configured to provide a body posture to be taken by a patient during a breast cancer diagnosis to the patient by a virtual object image that shows a body outline image for posture fitting, and a body fitting identifier 95 configured to calculate a degree of fitting between the body outline image of the patient and the body outline image for posture fitting and provide feedback to the patient.

In the present disclosure, it is preferred that the angle of incidence calculator 93 obtains depth information in three-dimensional coordinates for the ultrasound scanner by utilizing image sensors 30a and 30b that is disposed on the left and right sides to provide stereo vision, and calculates the position of the ultrasound scanner 100 and the angle of incidence of the ultrasound scanner.

In the present disclosure, it is preferred that a determination of the degree of fitting is calculated using any one of sum of squared difference (SSD), sum of absolute difference (SAD), K-nearest neighbor algorithm (KNN), and normalized cross correlation (NCC) techniques.

In addition, the smart mirror 700 includes a patient verification unit 91 configured to recognize who a patient is by comparing an image of a facial part on the body map with a face database of pre-registered patients. It is preferred that the controller 70 activates the thermographic camera mode, the ultrasound scanner mode, and the self-examination mode only for patients whose faces have been recognized.

Another aspect of the patient verification unit 91 is that a patient is authenticated by entering a fingerprint or ID number. It is preferred that the controller 70 activates the thermographic camera mode, the ultrasound scanner mode, and the self-examination mode only for the authenticated patient.

It is preferred that the image sensors 50a and 50b are installed through openings (not illustrated) prepared on the display panel 20b and the mirror 20a.

The thermographic camera 200 may be installed within the mirror 20a. In this case, it is preferred that a mirror film of the mirror 20a is processed with a material selected from germanium, chalcogenide, and zinc Serenide that allow light in the infrared band to pass through well so that the thermographic camera 200 may sense infrared light well.

A reference numeral 55 is a power supplier that supplies electricity to each part of the smart mirror 700.

The breast cancer tracking and management unit 72 according to an embodiment of the present disclosure may collect breast risk information of a patient according to the size, shape of a tumor, mass or calcification cluster obtained by the ultrasound artificial intelligence neural network 41 during the ultrasound scanner mode, and notify the patient by adjusting a schedule of the next breast cancer ultrasound examination according to a risk level of breast cancer of the patient, and determine the risk level of breast cancer by observing a trend over time through a periodic ultrasound follow-up examination for the patient accordingly.

It is preferred that the breast cancer tracking and management unit 72 collects the breast risk information of a patient obtained from the ultrasound artificial intelligence neural network 41 while the ultrasound scanner is scanning the breast hot spot area and utilizes the breast risk information for ultrasound follow-up.

In addition, it is preferred that the breast cancer tracking and management unit 72 additionally registers an area suspected of being a tumor, mass or calcification cluster by the ultrasound artificial intelligence neural network 41 during the ultrasound scanner mode as a breast cancer hot spot in the breast cancer hot spot memory 84, and includes the added breast cancer hot spot in the periodic ultrasound follow-up examination.

Further, the breast cancer tracking and management unit 72 may observe a trend of changes in the size and number of breast cancer hot spots according to the periodic follow-up examination by the thermographic camera 200 to inform the patient of a risk level of breast cancer or to inform the patient of a schedule for the next breast cancer thermographic camera examination.

In addition, the smart mirror 700 may further include a communication means (not illustrated) that provides Wi-Fi, Bluetooth connectivity, wired or wireless internet, and the Internet of Things.

In addition, the controller 70 performs a function of controlling the virtual object imaging unit 88 and the speaker 60 according to operations of the breast cancer tracking and management unit 72, the body fitting identifier 95, and the ultrasound artificial intelligence neural network 41.

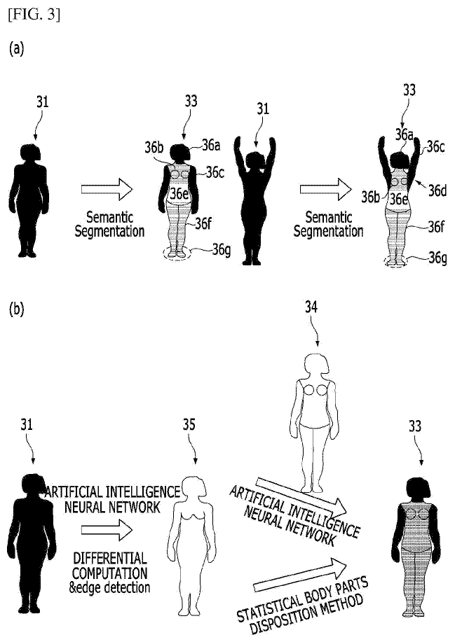

FIG. 3 illustrates several embodiments of a body navigator 90 configured to obtain a body map 33 from a body image 31. In (a) of FIG. 3, the body navigator 90 is implemented by obtaining the body map 33 by an artificial intelligence neural network that has been learned by the body image 31 labeled with body parts including a face 36a, breast 36b, arms 36c, armpits 36d, belly 36e, legs 36f, and feet 37g.

For example, when there is a need to examine the armpits 34d of a patient during a breast cancer diagnosis, the body navigator 90 may determine a position of the armpits 34d by examining a body map obtained by semantic segmentation of the body image 31 of the patient.

It is preferred that the virtual object image of the present disclosure includes boundary lines that distinguish virtual body parts.

The virtual body parts include the breast outline and the body outline of the patient.

FIG. 3B illustrates another embodiment of the body navigator 90, which may obtain a body outline image 35 from the body image 31, and obtain the body map 33 by labeling body parts on the body outline image 35 with a statistical body part disposition method based on the medical and physical disposition of the body parts (face, arms, breast, stomach, legs, feet) and a size ratio between the body parts.

Further, another aspect of FIG. 3B illustrates an embodiment in which the body outline image 35 is obtained from the body image 31 and the body map 33 is obtained by the artificial intelligence neural network that has been learned by the images labeled with the body outline images 34, which include boundary lines separating the body parts.

The body outline image 35 in FIG. 3B is obtained such that the artificial intelligence neural network is learned by the images labeled with the body outline image for the given body image 31, and then the body outline image is obtained using the learned artificial intelligence neural network for the given body image 31 of a patient from the image sensor.

In another aspect of the body outline image 35 of FIG. 3B, a differential image may be obtained by performing a differential calculation between an image when a patient is not in front of the smart mirror 700 and an image when the patient is in front of the smart mirror 700, and a border edge component of the differential image may be taken to obtain the body outline image 35.

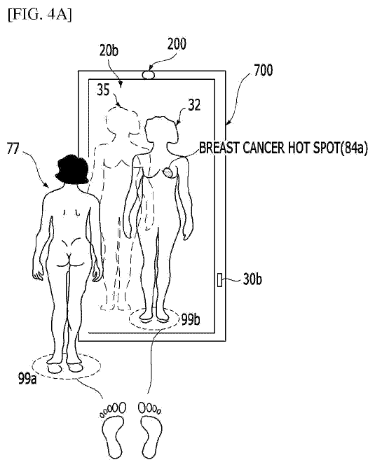

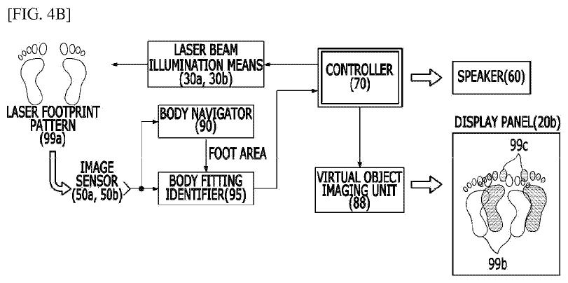

FIG. 4A illustrates various embodiments of a standing position information providing means (not illustrated) configured to provide position information to a patient 77 on an optimal standing place that is advantageous for diagnosing breast cancer when the patient 77 is standing in front of the smart mirror 700.

The standing position information providing means may be implemented by measuring a height of a patient by the image sensor 50a and 50b to calculate a standing position that is favorable for diagnosing breast cancer, and by performing a laser beam illumination of a laser footprint pattern 99a on the corresponding floor position with the laser beam illumination means 30a and 30b.

For example, the laser footprint pattern 99a in the shape of a footprint may be formed on a floor surface on which the smart mirror 700 is installed by the laser beam illumination means 30a, 30b to visually provide footprint position information to the patient 77.

In another aspect, the standing position information providing means may be implemented by displaying the body outline image for posture fitting 32 or the footprint outline for fitting 99b as an object image on the display panel 20b by using the virtual object imaging unit 88.

The body outline image for posture fitting 32 is the body outline image 35 of a patient that is displayed on the display panel 20b with the standing place as the origin coordinate. In this case, it is preferred that the body fitting identifier 95 calculates the degree of fitting between the body outline image 35 and the body outline image for posture fitting 32 based on the coordinates where a patient is currently standing and provides feedback to the patient by the controller 70.

The controller 70 provides a fitting guide to a patient by means of the speaker 60 of the smart mirror and the virtual object image on the display panel 20b.

It is preferred that the standing position information providing means provides the patient with the same standing position during the ultrasound scanner mode or self-examination mode as was used in the thermographic camera mode.

FIG. 4B is another embodiment of the standing position information providing means using the body fitting identifier 95 to identify whether a patient's sole 99c is within the range of the laser footprint pattern 99a by the image sensor 50a and 50b, that is, to identify how well the fitting is achieved.

A reference numeral 99b is a footprint outline for fitting 99b, which is a virtual object image of the laser footprint pattern 99a generated by laser illumination, or a virtual object image of the footprint shape at the desired standing position displayed on the display panel.

A reference numeral 99c is a patient sole pattern 99c, which is a virtual object image of a patient's foot 36g.

A difference in distance between the actual patient's foot 36g and the laser footprint pattern 99a may be calculated by the image sensors 50a and 50b that provide stereo vision, and the difference in distance may be represented on the display panel 20b as the footprint outline for fitting 99b and the patient sole pattern 99c by means of a virtual object image unit 88.

It is preferred that the body fitting identifier 95 calculates the degree of fitting between the footprint outline for fitting 99b and the patient sole pattern 99c and provides feedback to a patient by the controller 70. In this case, the controller 70 provides a fitting guide to the patient by means of the speaker 60 of the smart mirror and the virtual object image on the display panel 20b.

In addition, it is preferred that the laser footprint pattern 99a blinks when the patient's foot 36g remains outside the range of the laser footprint pattern 99a.

It is preferred that the controller 70 according to the present disclosure activates the thermographic camera mode, the ultrasound scanner mode, and the self-examination mode only for a patient who has been authenticated and whose body outline image 35 has been properly fitted with the body outline image for posture fitting 32 in the standing position.

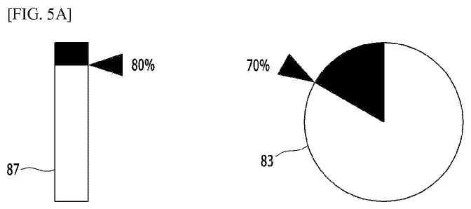

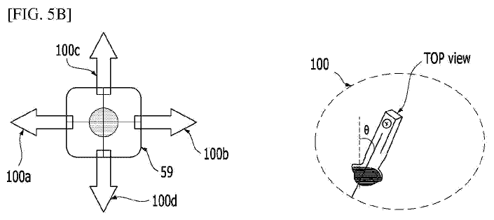

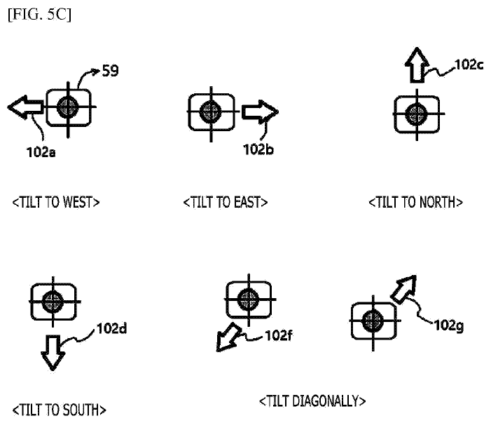

FIGS. 5A to 5C illustrate various embodiments of a virtual object image that represents a virtual cursor 59, pressure correction information, and incident angle correction information during the ultrasound scanner mode.

FIG. 5A illustrates an embodiment of pressure correction information that displays a required contact pressure information and a contact pressure of the current ultrasound scanner as an object image with a bar graph 87, pie graph 83, or numerical value.

The pressure correction information displayed on the mirror 20a serves as a reference for a patient when correcting a pressure in the ultrasound scanner 100.

FIGS. 5b and 5c are an embodiment of a virtual object image that incorporates a virtual cursor 59 and angle of incidence correction arrows 100a,100b, 100c, and 100d to indicate the east-west and north-south directions of the angle of incidence correction arrows when viewed from the top of the ultrasound scanner 100.

That is, in this embodiment, a virtual object image represented by the angle of incidence correction arrows 100a, 100b, 100c, and 100d, together with the virtual cursor 59, indicating the east-west and north-south directions when viewed from the top of the ultrasound scanner 100, is provided to the patient by the smart mirror 700. In this case, the patient may obtain incident angle correction information by the angle of incidence correction arrow and identify a position of the current ultrasound scanner 100 by a position of the virtual cursor 59.

FIG. 5C is an embodiment of an object image in which a position and an incident angle of the ultrasound scanner 100 are identified by the image sensor 50a and 50b and the body posture interaction unit 51. The angle of incidence correction arrows 100a, 100b, 100c, and 100d that are overlapped on an image of the ultrasound scanner 100 reflected in a mirror are incident angle correction information, which is referenced for correcting the incident angle of the ultrasound scanner to a patient.

In addition, the virtual cursor that is overlapped on an image of the ultrasound scanner 100 reflected in the mirror is “position information”, which is referenced for correcting a position of the ultrasound scanner to a patient.

It is preferred that the incident angle correction information of the ultrasound scanner 100 utilizes an angle of incidence correction arrow that includes upward, downward, and left-right directions. The incident angle correction information is a virtual object image that instructs a person performing self-diagnosis, using arrow directional instructions, how to reach a pre-empirically known ideal incident angle of the ultrasound scanner 100 according to the examination site.

When the incident angle of the current ultrasound scanner is inconsistent with the required incident angle, it is preferred that an angle of incidence correction arrow in a direction that requires correction is displayed as an object image by blinking on the smart mirror to guide the patient to correct the incident angle.

For example, when the ultrasound scanner 100 needs to be tilted in the west direction, a west angle of incidence correction arrow 102a is displayed or blinked.

A reference numeral 102b is an east angle of incidence correction arrow, a reference numeral 102c is a north angle of incidence correction arrow, a reference numeral 102d is a south angle of incidence correction arrow, and reference numerals 102f and 102g are diagonal angle of incidence correction arrows.

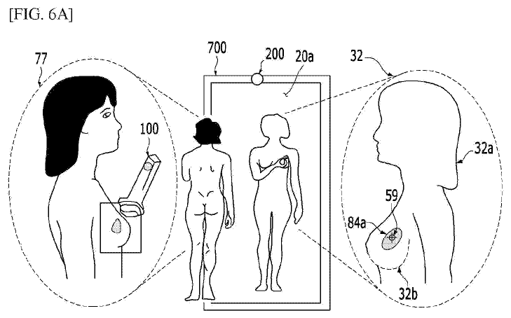

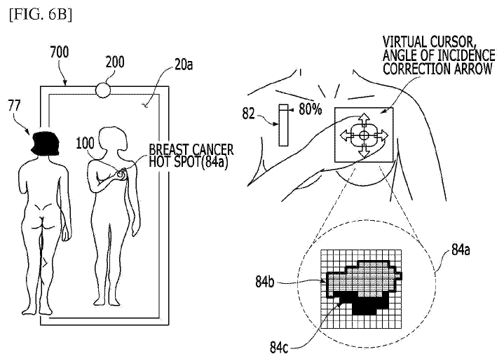

FIGS. 6A to 6B illustrate embodiments of performing ultrasound self-diagnosis using the smart mirror 700 to overlap a virtual object image required for breast cancer diagnosis on a patient 77's own image reflected in the mirror 20A on the display panel 20B.

A virtual cursor 59 indicates the current position of the ultrasound scanner 100 on the patient's own reflection in the mirror during the ultrasound scanner mode.

FIG. 6A is an embodiment of re-examination using the ultrasound scanner 100 for the breast cancer hot spot 84a area obtained in the thermographic camera mode. The breast cancer ultrasound self-diagnosis is performed while identifying the current position of the ultrasound scanner 100 by the virtual cursor 59 shown on the display panel 20B. The body outline image 32 includes a breast outline 32b and a body outline 32a.

In showing the patient 77 the breast cancer hot spot 84a obtained in the thermographic camera mode and the virtual cursor 59 indicating the current position of the ultrasound scanner 100 in an object image, the patient 77 may easily identify the current position of the ultrasound scanner 100 and understand which direction to move the ultrasound scanner 100 to reach an area of the breast cancer hot spot 84a.

The hot spot guider 86, during the ultrasound scanner mode, displays the area and position of the breast cancer hot spot 84a obtained in the thermographic camera mode, relative to the breast outline 32b, overlapped on a patient's breast reflected in a mirror through an object image in augmented reality.

In addition, it is preferred that the breast cancer tracking and management unit 72 informs a patient of a risk level of breast cancer or a schedule for the next breast cancer examination by displaying an object image or text message on the display panel 20b of the smart mirror 700.

FIG. 6B is another embodiment of re-examining an area of breast cancer hot spot 84a obtained in thermographic camera mode using the ultrasound scanner 100. The ultrasound self-diagnosis is performed while identifying the current position and angle of incidence of the ultrasound scanner 100 by a virtual cursor and an angle of incidence correction arrow shown on the display panel 20B.

The pressure correction information 82 indicates how much pressure the ultrasound scanner is applying to the affected area as a pressure level compared to a standardized pressure.

It is preferred that the hot spot guider 86 according to embodiments of the present disclosure displays a breast hot spot area 84b that has been re-examined by the ultrasound scanner 100 in a blue color, and a breast hot spot area 84c that has not been re-examined by the ultrasound scanner 100 in a red color.

Hereinafter, an operation flow of the present disclosure will be briefly described, based on the details described above. Although not illustrated in the drawings, a method of self-diagnosing breast cancer may be performed by the above described artificial intelligence thermographic ultrasound scanner device. Hereinafter, for convenience of explanation, the method will be described as being performed by the artificially intelligent thermographic ultrasound scanner device.

In step S100, an optimal position information favorable for diagnosing breast cancer may be provided to a patient by the standing position information providing means.

In step S101, a breast cancer hot spot may be found by the thermographic camera.

In step S102, when a breast cancer hot spot is detected by the thermographic camera, the patient may be requested to perform the ultrasound examination or self-examination.

In step S103, posture correction information of the ultrasound scanner may be provided as a virtual object image on the smart mirror during the ultrasound scanner mode.

In step S104, the breast cancer hot spot found during the self-diagnosis may be displayed on the smart mirror by overlapping the breast cancer hot spot with an image of the patient reflected in the mirror in augmented reality.

In step S105, periodic ultrasound follow-up examinations may be performed by the breast cancer tracking and management unit to observe changes in the size of a tumor, lump, or calcification cluster over time to inform the patient of a risk of breast cancer or a schedule for the next breast cancer examination.

In step S106, the size and number of breast cancer hot spots may be observed by the breast cancer tracking and management unit according to the periodic follow-up examinations by the thermographic camera to inform the patient of a risk of breast cancer or a schedule for the next breast cancer examination.

In the above-mentioned description, steps S100 to S106 may be divided into additional steps or combined into fewer steps according to the embodiment of the present disclosure. In addition, some steps may be omitted as needed, and the order between the steps may vary.

The method of self-diagnosing breast cancer according to the embodiment of the present application may be implemented in the form of program commands executable by means of various computer means and then written in a computer-readable recording medium. The computer-readable medium may include program instructions, data files, data structures, or the like, in a stand-alone form or in a combination thereof.

The program instructions recorded in the medium may be specially designed and configured for the present disclosure or may be known and available to those skilled in computer software. Examples of the computer-readable recording medium may include magnetic media, such as a hard disk, a floppy disk and a magnetic tape, optical media, such as CD-ROM and DVD, magneto-optical media, such as a floptical disk, and hardware devices, such as ROM, RAM and flash memory, which are specifically configured to store and run program instructions. Examples of the program instructions may include machine codes made by, for example, a compiler, as well as high-language codes that may be executed by an electronic data processing device, for example, a computer, by using an interpreter. The above-mentioned hardware devices may be configured to operate as one or more software modules in order to perform the operation of the present disclosure, and the opposite is also possible.

In addition, the method of self-diagnosing breast cancer has been described above may also be implemented in the form of a computer program or application stored in a recording medium and executed by a computer.

It will be appreciated that the embodiments of the present application have been described above for purposes of illustration, and those skilled in the art may understand that the present application may be easily modified in other specific forms without changing the technical spirit or the essential features of the present application. Therefore, it should be understood that the above-described embodiments are illustrative in all aspects and do not limit the present disclosure. For example, each component described as a single type may be carried out in a distributed manner. Likewise, components described as a distributed type can be carried out in a combined type.

The scope of the present application is represented by the claims to be described below rather than the detailed description, and it should be interpreted that the meaning and scope of the claims and all the changes or modified forms derived from the equivalent concepts thereto fall within the scope of the present application.
20a: Mirror
20b: Display panel
30a and 30b: Image sensor (laser beam illumination means)
31: Body image
32: Body outline image for posture fitting
32a: Body outline
32b: Breast outline
33: Body map
35: Body outline image
36a: Face
36b: Breast
36c: Arms
36d: Armpit
36e: Belly
36f: Legs
36g: Foot
40: Wireless Receiver
41: Ultrasound artificial intelligence neural network
43: Pressure sensing artificial intelligence neural network
50a: Image sensor
50b: Image sensor
51: Body posture interaction
52: Thermographic breast cancer detector
55: Power supplier
59: Virtual cursor
60: Speaker
70: Controller
72: Breast cancer tracking and management unit
77: Patient
80: Breast thermographic image mapper
82: Cut-off value adjuster
82: Pressure correction information
83: Pie graph
84: Breast cancer hot spot memory
84a: Breast cancer hot spot
84b: Breast hot spot area re-examined
84c: Breast hot spot area not re-examined
86: Hot spot guider
87: Bar graph
88: Virtual Object Imaging unit
90: Body navigator
91: Patient verification unit
92: Body posture correction requester
93: Angle of incidence calculator
95: body fitting identifier
99a: Laser footprint pattern
99b: Footprint outline for fitting
99c: Patient sole pattern
100: Ultrasound scanner
100a,100b,100c, and 100d: Angle of incidence correction arrows
200: Thermographic camera
420: Wireless transmitter
600: Thermographic ultrasound scanner device
700: Smart mirror

1. An artificial intelligence thermographic ultrasound scanner device comprising a smart mirror and an ultrasound scanner.

2. The artificial intelligence ultrasound scanner device of claim 1, diagnosing a disease is performed using the ultrasound scanner while observing an image of a patient reflected in a mirror of the smart mirror in a mode in which the ultrasound scanner is operating.

3. The artificial intelligence ultrasound scanner device of claim 1, wherein the smart mirror comprises:
a thermographic camera configured to detect thermal radiation emitted from a patient's body to obtain a two-dimensional thermographic image;
a plurality of image sensors configured to obtain a body image of the patient;
a wireless receiver configured to receive ultrasound image information collected from the ultrasound scanner;
a speaker configured to identify position information on the ultrasound scanner and inform the patient of feedback control commands or instructions required for an ultrasound diagnosis with a voice service;
a display panel configured to identify posture information on the ultrasound scanner and deliver the feedback control commands through a virtual object image to guide a posture of the ultrasound scanner by an inspection area, or to inform the instructions required for the ultrasound diagnosis through the virtual object image;
a virtual object imaging unit configured to generate the virtual object image on the display panel;
a thermographic breast cancer detector configured to detect a breast cancer hot spot, which is an area suspected of having breast cancer from the thermographic image; and
an ultrasound artificial intelligence neural network that has been pre-learned by breast cancer ultrasound images for training, labeled according to a rating of risk level of breast cancer and configured to determine signs of breast cancer from the ultrasound image information, and
wherein the smart mirror displays the virtual object image on the display panel with the virtual object image overlapped on an image of the patient reflected in the mirror, and analyzes the ultrasound image information obtained from the patient by the ultrasound artificial intelligence neural network to automatically determine the risk of breast cancer of the patient.

4. The artificial intelligence ultrasound scanner device of claim 1, wherein the ultrasound scanner comprises:
an ultrasound probe configured to obtain the ultrasound image information from the affected area in contact with the patient's breast; and
a wireless transmitter configured to transmit the ultrasound image information to the wireless receiver of the smart mirror.

5. The artificial intelligence ultrasound scanner device of claim 3, wherein the thermographic breast cancer detector comprises:
a breast thermographic image mapper configured to generate a breast thermographic image consisting of an average value of a pixel-by-pixel cumulative sum of thermographic images of a breast area taken equal to or more than a predetermined number of times over a predetermined period of time;
a cutoff value adjuster configured to generate a thermographic hot spot image comprising a breast cancer hot spot that represents a temperature value equal to or more than a predetermined value in the breast thermographic image; and
a breast cancer hot spot memory configured to store the thermographic hot spot image or breast cancer hot spot information.

6. The artificial intelligence ultrasound scanner device of claim 3, wherein the smart mirror further comprises a hot spot guider configured to inform an area and position of the breast cancer hot spot obtained from a thermographic camera mode during an ultrasound scanner mode or a self-examination mode in augmented reality by overlapping the object image to the mirror on which the patient's own image is projected.

7. The artificial intelligence ultrasound scanner device of claim 3, wherein the smart mirror further comprises a pressure sensing artificial intelligence neural network that has been pre-learned by breast ultrasound images labeled according to magnitudes of various pressure levels and determines a magnitude of a pressure level of the ultrasound scanner from breast ultrasound images obtained during an ultrasound scanner mode.

8. The artificial intelligence ultrasound scanner device of claim 3, wherein the smart mirror further comprises a body posture interaction unit, and
wherein the body posture interaction unit comprises:
an angle of incidence calculator 93 configured to calculate a position of the ultrasound scanner and a position and incident angle of the ultrasound scanner by the image sensor, and provide position and incident angle correction information of the ultrasound scanner;
a body navigator configured to create a body map which comprises boundary lines to distinguish body parts and a body outline from the body image;
a body posture correction requester configured to provide a body posture to be taken by the patient during a breast cancer diagnosis by a virtual object image configured to show a body outline image for posture fitting, a body fitting identifier configured to calculate the degree of fitting between the body outline image of the patient and the body outline image for posture fitting and provide feedback to the patient; and
a patient verification unit configured to verify who the patient is.

9. The artificial intelligence ultrasound scanner device of claim 8, wherein the patient verification unit is a face recognition unit that is configured to recognize who a face is by comparing an image of a facial part on the body map with a face database of pre-registered patients.

10. The artificial intelligence ultrasound scanner device of claim 8, wherein the body navigator comprises an artificial intelligence neural network that has been learned by deep learning from body images labeled with semantic segmentation in different colors for body parts, and obtains the body map using the artificial intelligence neural network for a body image of a given patient from the image sensor.

11. The artificial intelligence ultrasound scanner device of claim 8, wherein the body navigator comprises an artificial intelligence neural network that has been learned by deep learning from body images labeled with a body outline image that comprises boundary lines separating body parts, and obtains the body map using the artificial intelligence neural network for a body image of a given patient from the image sensor.

12. The artificial intelligence ultrasound scanner device of claim 3, wherein the smart mirror further comprises a standing position information providing means configured to calculate position information corresponding to a breast cancer diagnosis during a thermographic camera mode and provide the patient with the position information.

13. The artificial intelligence ultrasound scanner device of claim 12, wherein the position information providing means displays a body outline image for fitting or a footprint outline for fitting which indicates a standing place favorable for diagnosing breast cancer by measuring a height of the patient by the image sensor through the virtual object image on the display panel, or provides a standing place by a laser beam scanning means.

14. The artificial intelligence ultrasound scanner device of claim 3, wherein the virtual object image is any one or more object images selected from a virtual cursor, pressure correction information, incident angle correction information, a breast outline, a body outline, a footprint outline for fitting, boundary lines distinguishing body parts, and a breast cancer hot spot.

15. The artificial intelligence ultrasound scanner device of claim 6, wherein the hot spot guider, during an ultrasound scanner mode or a self-examination mode, displays the breast cancer hot spot on the display panel by overlapping the breast cancer hot spot on the patient's breast image reflected in the mirror in augmented reality to guide the patient to re-examine the breast cancer hot spot area.

16. The artificial intelligence ultrasound scanner device of claim 5, wherein the smart mirror further comprises a breast cancer tracking and management unit configured to perform periodic ultrasound follow-up examinations to observe changes in size of a tumor, lump, or calcification cluster over time to inform the patient of a risk level, or to additionally register a tumor, lump, or calcification cluster detected during an ultrasound scanner mode in the breast cancer hot spot memory, or to inform the patient of a schedule for the next breast cancer ultrasound examination.

17. The artificial intelligence thermographic ultrasound scanner device of claim 3, wherein the smart mirror further comprises a breast cancer tracking and management unit configured to observe changes in the size and number of breast cancer hot spots based on periodic follow-up examinations by the thermographic camera to inform the patient of a risk level or to inform the patient of a schedule for the next breast cancer thermographic camera examination.

18. A method of self-diagnosing breast cancer performed by the artificial intelligence thermographic ultrasound scanner device according to claim 1, the method comprising:
providing a patient with optimal position information favorable for diagnosing breast cancer by a standing position information providing means;
finding a breast cancer hot spot by a thermographic camera;
requesting an ultrasound examination or self-examination when the breast cancer hot spot is detected by the thermographic camera;
providing posture correction information on the ultrasound scanner with a virtual object image on the smart mirror during an ultrasound scanner mode; di splaying the breast cancer hot spot found during self-diagnosis on the smart mirror by overlapping the breast cancer hot spot on an image of a patient reflected in the mirror in augmented reality;
performing periodic ultrasound follow-up examinations by a breast cancer tracking and management unit to observe changes in size of a tumor, lump, or calcification cluster over time to inform the patient of a risk level of breast cancer or a schedule for the next breast cancer examination; and
observing changes in the size and number of breast cancer hot spots based on the periodic follow-up examinations with the thermographic camera by the breast cancer tracking and management unit to inform the patient of a risk of breast cancer or a schedule for the next breast cancer examination.
